(19) United States
(12) Patent Application Publication
(10) Pub. No.: US 2018/0249263 A1
(43) Pub. Date: Aug. 30, 2018

(54) Playing Modified Audio Signals to a Group of Listeners

(71) Applicant: MEQ INC., San Jose, CA (US)

(72) Inventors: Ofer RAZ, Albany, CA (US); Danny ARONSON, Tel Aviv (IL)

(21) Appl. No.: 15/751,988

(22) PCT Filed: Jul. 14, 2016
(86) PCT No.: PCT/IL2016/000013

§ 371 Date: Feb. 12, 2018

Related U.S. Application Data
(60) Provisional application No. 62/204,472, filed on Aug. 13, 2015.

(51) Int. Cl. H04R 25/00; A61B 5/12

(52) U.S. Cl. CPC H04R 25/70; A61B 5/121; H04R 25/30; H04R 25/353

(57) ABSTRACT

A method and a device are provided for modifying audio signals for a group of listeners in accordance with hearing capabilities of at least two members belonging to that group. The method comprises the steps of: providing information that relates to hearing capabilities of the at least two members; based on the information provided, determining respective values for one or more parameters to be applied for modifying audio signals that are about to be played, in a way that would suit all members belonging to that group of listeners; modifying the audio signals by applying the respective values of the determined parameters to modify audio signals; and enabling playing the modified audio signals to all members of the group of listeners.

### TECHNICAL FIELD

The present disclosure relates to the field of consumer electronics, and in particular to devices that enable listening to audio signals.

### BACKGROUND

Hearing capabilities change from one person to the other, hence audio signals to which the users are able to listen, change from one person to the other, depending primarily on one's hearing capabilities. The term “hearing range” usually describes the range of frequencies that can be heard by humans, though it can also refer to the range of levels. The human hearing range is commonly given as 20 to 20,000 Hz, but there is a considerable variation between different individuals, especially at high frequencies. A gradual decline with age is considered normal, and sensitivity also varies with frequency. Individual hearing range varies according to the general condition of the individual's ears. The range shrinks during life, usually beginning at around age of eight with the upper frequency limit being lowered. Women typically experience a lesser degree of hearing loss than men, with a later onset. Men have approximately 5 to 10 dB greater loss at the upper frequencies by the age of 40. On top of that, there may also be substantial differences between one's hearing capabilities in his/her left and right ears.

In view of the above, it is clear that the listening experience of a listener could be enhanced when audio signals to which he/she listens, are modified in a way that fits that individual. Certain attempts to provide solutions for carrying out this idea have been made, among which solutions that were proposed by the present applicants in their application published under WO 2016/020908, application No. PCT/IL2016/000006 filed on Apr. 12, 2016, application No. PCT/IL2016/000012 filed on Jun. 16, 2016, as well as by others.

However, all these and other methods aim to improve the audio quality and listening experience of a single person. In other words, these methods are adapted to enable an individual to undergo a certain hearing test and then are configured to use the results obtained, for modifying audio signals by applying a certain compensation based on the results of the individual profile (e.g. the individual's hearing deterioration profile at certain frequencies (audiogram) for each ear). However, it is rather obvious that none of these methods may be used to provide an adequate solution when the audio signals are played by a music player to a group of individuals listening to that music player. If any method known in the art is to be implemented in such a case, the audio signals would have been modified in accordance with the hearing profile of one individual who is a member of the group of people listening to the music, so that specific individual would obviously enjoy an improved audio experience, but at the same time, for any other individual of that group who listens to the audio signals modified in accordance with the hearing profile of that first specific individual, the modified audio signals might sound out of balance and irritating.

Thus, if one were to take the concept of modifying audio signals based on the individual hearing capabilities a step further, from personalized audio systems where the modified signals are adapted only for the individual who had undergone the hearing test (e.g. systems such as headphones or personal speakers), to a shared environment (for example when listening to music being played in a car or by home audio systems), where few people may be listening at the same time to audio signals being played by a single source, all prior art solutions would fail to provide an adequate answer, as they are not adapted to modify audio signals in a way that they will sound pleasant to all members in that shared audio environment.

### SUMMARY OF THE DISCLOSURE

The disclosure may be summarized by referring to the appended claims.

It is an object of the present disclosure to provide a novel method and device adapted to modify audio signals in accordance with hearing capabilities of a group of individuals who are listening to the audio signals when played by a single source.

It is another object of the present disclosure to provide a method and device for obtaining and storing a plurality of audiograms each associated with a respective individual, and to enable modifying audio signals when a group of individuals listen to the audio signals, where the modification is carried out in a way that is based on information retrieved from the respective audiograms of the group members.

It is yet another object of the present disclosure, to provide a method for obtaining instructions on how should audio signals be modified every time when a group of certain individuals is listening to these modified audio signals, and preferably, irrespective of the device through which these modified audio signals are played.

It is another object of the present disclosure, to provide a method for storing information (e.g. information associated with an audiogram) on how should audio signals be modified when an individual joins a group of individuals who are listening to the modified audio signals.

It is another object of the present disclosure to provide a method for obtaining and storing a plurality of audiograms each associated with a respective individual, and to enable modifying audio signals to be played to a group of members selected from among these individuals.

It is still another object of the present disclosure to provide a method for modifying audio signals in accordance with hearing capabilities of members of a group comprising specific individuals who are listening to the audio signals, based on information retrieved from a plurality of hearing tests that the group members had undergone.

It is another object of the present disclosure to provide a method and system for remotely storing information that relates to hearing capabilities of each member belonging to a group of individuals, and when that group of individuals wishes to listen to a musical creation played by a music player, the information may be retrieved and forwarded to the music player, which then modifies the audio signals in accordance with the hearing capabilities of these individuals, prior to the playing these audio signals.

Other objects of the present invention will become apparent from the following description.

According to a first embodiment of the present disclosure, there is provided a method for modifying audio signals for a group of listeners in accordance with hearing capabilities of at least two members belonging to a group of individuals/listeners (e.g. people who wish to listen together to audio signals played by a device adapted to generate audio signals), the method comprises the steps of:

providing information that relates to hearing capabilities of the at least two members belonging to a group of individuals/listeners, for example, results of one or more hearing tests taken by the at least two group members, information derived from one or more hearing tests taken by the at least two group members, deviations in the hearing capabilities of the at least two group members from a pre-defined hearing pattern, instructions on how to modify tones (e.g. the compensation required for each of the at least two members), based on the respective hearing capabilities of the at least two members, etc.;

based on the information provided, determining respective values for one or more parameters to be applied for modifying audio signals that are about to be played in a way that may suit all members belonging to the group of listeners (e.g. values for one or more parameters that is associated with a common audiogram);

modifying the audio signals by taking into consideration the respective values of the one or more determined parameters; and

enabling playing the modified audio signals to members of the group of listeners.

The term “hearing test” as used herein throughout the specification and claims is used to denote a test carried out to determine hearing capabilities of one or both ears of an individual. The hearing test may be conducted as two separate tests, each for a different ear of the individual, or as one test for determining the combined individual's hearing capabilities when both ears are being subjected simultaneously to the hearing test.

The term “hearing capabilities” as used herein throughout the specification and claims is used preferably but not exclusively to denote gaps that exist between tones as they may be heard by an individual who listens to audio tones, and as these audio tones should have been heard by the individual, had he/she had a perfect hearing ability. For the same individual, the gap may be different at different frequencies (or at different ranges of frequencies) and may vary between one of the individual's ears and the other. These gaps, providing a characterization of the hearing capabilities of each of the at least two listeners, may be taken into account when establishing how should the audio tones be modified, when they will be played to a group that comprises the at least two listeners. However, it should be noted that when combining information derived from hearing tests conducted to the at least two listeners, the way that the audio signals will eventually be modified, is not necessarily one that would overcome all gaps existing in the hearing capabilities of the at least two members (and obviously it is not necessarily a way that would overcome all gaps in the hearing capabilities of all members who belong to the group), but rather a way that would enable all the members of the group to enjoy listening to the audio signals in their modified form, in a way that will sound pleasant to them.

When reference is made throughout the specification and claims to information that relates to the hearing capabilities of an individual, it should be understood to encompass each of the following options or any combination thereof: the results of one or more hearing tests taken by that individual, and/or any data derived from the results of one or more hearing tests, e.g. data that would enable taking into account the individual's hearing capabilities when determining how to modify the audio signals in a way that will be suited for all members of the group (such as compensation related data).

When reference is made throughout the specification and claims to “a group of listeners”, it should be understood to relate to a group of people (individuals) who wish to listen together to audio signals (e.g. music), where the audio signals are generated by a single source, irrespective whether they are already listening to the audio signals, or will be listening to them when they are played at a later stage. Also, it should be understood that not all members of the group of listeners must be located at the same geographical location, however, in order to qualify as members of a certain group of listeners, they should all listen at the same time to the same audio signals, when generated by the same audio signals' generator.

According to another embodiment of the disclosure, the information that relates to the hearing capabilities of the at least two members belonging to a group of listeners, comprises deviations in the hearing capabilities of the at least two listeners from a pre-defined hearing pattern.

In accordance with another embodiment, the step of determining the one or more parameters for modifying audio signals comprises averaging gaps associated with a plurality of audio tones that exist between hearing capabilities of each of the at least two members of the group and the plurality of audio tones as they should have been heard by these at least two members.

By yet another embodiment, the method further comprises a step of selecting on an ad hoc basis who will be members of the group of listeners, prior to playing the modified audio signals to the listeners who would eventually belong to that group.

According to still another embodiment, at least one of the members of the group of listeners is a member of at least one other group of listeners.

According to another embodiment, the step of providing information comprises providing information that relates to hearing capabilities of a first plurality of members belonging to the group of listeners, and determining the one or more parameters for modifying audio signals based on information that relates to hearing capabilities of a second plurality of members, wherein the second plurality of members is selected from among the first plurality of members. This way, the hearing capabilities of some of the members of the group (belonging to the second plurality) may influence the modification of the audio signals when played to the group, while at the same time, the hearing capabilities of other members of that group, will not influence the modification, or in the alternative, will influence the modification substantially less than the hearing capabilities of the members that belong to the second plurality of members.

In accordance with another embodiment, at least one of the one or more parameters has at least two different values, each associated with a respective range of frequencies.

By yet another embodiment, the step of providing information comprises providing information that relates to hearing capabilities of each ear of the at least two members who belong to the group of listeners.

According to still another embodiment, the method further comprises a step of determining at least one range of frequencies at which input audio signals will be modified, and how to modify input audio signals belonging to that range of frequencies.

According to another aspect of the disclosure, there is provided a music player configured to modify audio signals in accordance with hearing capabilities of at least two members belonging to a group of listeners (e.g. individuals who may wish to listen together to audio signals being played by the music player, such as a musical creation), wherein the music player comprising:

at least one processor configured to:
(i) receive information that relates to hearing capabilities of the at least two listeners who are members belonging to the group of listeners; and
(ii) modify audio signals to be played to that group of listeners, by taking into account the received information, in a way that would suit all members belonging to that group of listeners;

a sound generating module configured to play the modified audio signals.

The term “music player” as used herein throughout the specification and claims, is used to denote any device that is provided with means to play audio signals (e.g. a media player, a music center, etc.).

According to another embodiment of this aspect of the disclosure, the at least one processor is further configured to determine, based on the information received, respective values for one or more parameters to be applied while modifying audio signals that are about to be played, wherein the determination is made in a way that would suit all members belonging to that group of listeners.

In accordance with another embodiment, the music player further comprising a receiver configured to receive information from another electronic device, wherein the information relates to the hearing capabilities of the at least two members of the group of listeners. It should be noted that the term “another electronic device” should be understood any device that is capable of forwarding that information to the music player in an electronic form such as for example, a remote server, a smartphone, a computer, a tablet, a PDA, and the like. Preferably but not necessarily, the receiver is operative to receive information that is wirelessly transmitted thereto.

By yet another embodiment, the other electronic device is a server located remotely from the music player (e.g. implementing a cloud storage), and configured to store the information that relates to the hearing capabilities of the at least two members of the group of listeners.

The term “located remotely” which relates to the electronic device described herein, is used throughout the specification and claims to denote an electronic device that is placed/installed at a different geographical location.

According to still another embodiment, the music player further comprises a user interface configured to enable receiving a command initiated by or on behalf of at least one listener, wherein the command comprises an identification of at least one listener who belongs to the group of listeners or an identification of the group itself.

In accordance with another embodiment, the music player further comprises a transmitter operative to transmit a command received by the music player from or on behalf of one or more members of the group of listeners, towards the other electronic device, instructing the other electronic device to forward the information that relates to hearing capabilities of the at least two members of the group of listeners, towards the music player.

According to another embodiment, the music player further comprises a storage configured to store information that relates to hearing capabilities of the at least two members of the group of listeners. In accordance with this embodiment, the processor may further be configured to retrieve information that relates to hearing capabilities of the at least two members of the group of listeners from the storage, and utilize it for modifying the audio signals that are about to be played to the group of listeners.

According to another embodiment, the information that relates to the hearing capabilities of the at least two group members comprises data which relates to at least one ear of each of the two group members and at least one frequency at which the audio signal will be modified, and wherein the modification will be affected to a portion of the audio signal associated with that at least one frequency.

In accordance with another embodiment, the information that relates to the hearing capabilities of the at least two group members, comprises information that enables the at least one processor to determine a comfortable volume baseline for all members of the group.

By yet another aspect there is provided a computer program product encoding a computer program stored on a non-transitory computer-readable medium for executing a set of instructions by one or more computer processors for establishing a process for carrying out the process described in the present disclosure.

### BRIEF DESCRIPTION OF THE DRAWINGS

For a more complete understanding of the present invention, reference is now made to the following detailed description taken in conjunction with the accompanying drawings wherein:

FIG. 1—is a flow diagram exemplifying a method carried out in accordance with an embodiment of the present invention; and

FIG. 2—is an exemplified block diagram presenting a system and optional connections between its components for implementing an embodiment of the present invention.

### DETAILED DESCRIPTION

In this disclosure, the term “comprising” is intended to have an open-ended meaning so that when a first element is stated as comprising a second element, the first element may also include one or more other elements that are not necessarily identified or described herein, or recited in the claims.

In the following description, for the purposes of explanation, numerous specific details are set forth in order to provide a better understanding of the present invention by way of examples. It should be apparent, however, that the present invention may be practiced without these specific details.

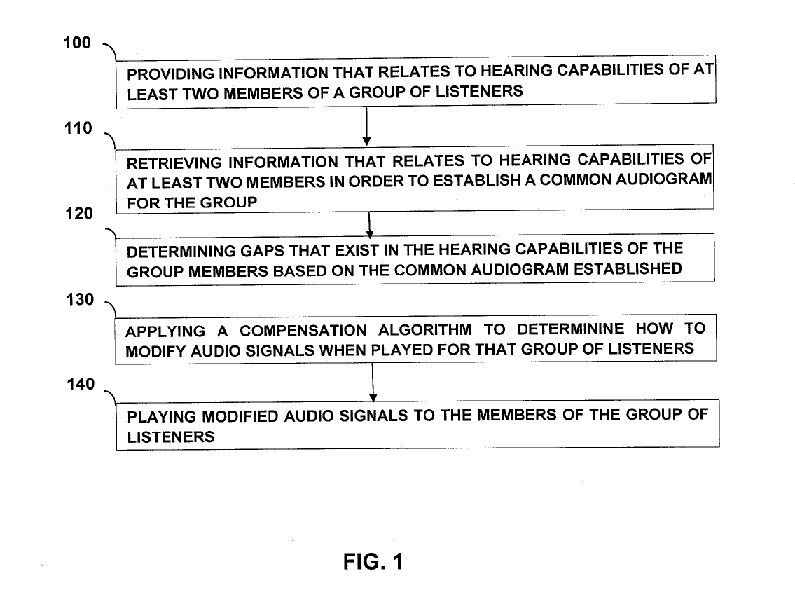

FIG. 1 is a flow diagram being a non-limiting example of a method carried out in accordance with an embodiment of the present invention.

In step 100 information that relates to hearing capabilities of two members (or more) who belong to a group of individuals such as a family, a party of friends etc., is provided. This information may be derived for example from hearing tests taken by each of the two members and is typically performed for each of the individual's ears, separately. The results of such hearing tests for each member are two distinct audiograms, one for the left ear and the other for the right ear.

The term “audiogram” as used herein, is used to denote a graph that shows the audible threshold for frequencies as measured by an audiometer. The Y axis represents intensity measured in decibels while the X axis represents frequency measured in Hertz. The threshold of hearing is plotted relative to a standardized curve that represents normal hearing, in dB.

Typically, hearing thresholds of humans are found by using hearing tests which involve different tones being presented at a specific frequency (pitch) and intensity (loudness). When the person hears the sound he/she presses a button so that the testing means recognizes that that tone was heard. The lowest intensity sound one can hear at each frequency being tested, would be recorded.

The hearing tests for obtaining the information that relates to the hearing capabilities of the at least two group members, may be carried out by using any type of applicable means. One option, is by using a set of headphones, comprising a DSP component having a software embedded thereon, which allows an individual to go through a hearing test. Another option is to utilize results obtained externally to the system (e.g. results obtained in a test carried out by a professional audiologist).

Optionally, the test that is carried by each of the two members differs from standard hearing tests as it may be designed to test primarily frequencies that pertain to music tones, and is thus different from conventional tests that focus mainly on frequencies that make speech more comprehensible, for those experiencing a hearing loss.

After completing the test, the results are stored. In the present example, the results are uploaded to a remote server and are stored thereat (e.g. using cloud storage). Uploading the results to the remote server may be done by any applicable way, such as for example the following one: following completion of the hearing test, the individual activates a dedicated application stored on his/her smartphone (or any other applicable device) which in turn establishes a connection (e.g. by using a Wi-Fi connection, a Bluetooth connection, a wired connection and the like) with the set of headphones. The data which relates to the individual's hearing test results, is then downloaded to his/her smartphone, followed by establishing a connection (e.g. via the Internet) between the smartphone and a remote server, for uploading data from the smartphone to the remote server.

Such a process (or any other process designed for obtaining a similar result) is carried out for each of the two (or more) members of the group, ending up by storing data that characterizes the hearing capabilities of each of these group members at the remote device (e.g. the remote server).

As already mentioned hereinabove, the method provided by the present invention is configured to allow modifying audio signals in accordance with the hearing capabilities of a number of individuals being members of a group of people who are about to listen to music comprising the audio signals that will be modified. However, in order to carry out such modifications, the data retrieved in the hearing tests are processed so that eventually they can be translated into a form that would enable appropriate modifications of the audio signals, e.g. modifying the audio signals depending on their frequencies (or frequency ranges/bands to which they belong). Such a translation may involve for example determining deviations that exist between the hearing capabilities of each of these members and a pre-defined hearing pattern. As will be appreciated by those skilled in the art, the processing of the hearing tests' results may be carried out at any device along a chain of devices that comprises: the set of headphones, the smartphone, the remote server and the music player, depending on various considerations, such as the impact of adding processing capabilities and/or storage capacity to the device price. The present invention should be understood to encompass all these options.

Next, when the members of the group get together, e.g. say a group of friends who intend to drive together in a car, according to the present example, the group members whose data that relates to their hearing capabilities are already stored at the remote device, initiate retrieval of their respective data from the remote device for processing them as part of creating a group combination (e.g. a common audiogram) (step 110). This step may be carried out in different variations. For example, one option is that one or more messages are sent to the remote device, indicating the members (who have their characterizing data stored thereat) who temporarily belong to the current group. Once it has been established who are the group members whose relevant information (e.g. personal audiograms) has already been stored at the remote device, a processor is configured to process the available data in order to generate new data (e.g. a new audiogram) to enable determining of what should be the common compensation for the gaps that exist in the hearing capabilities of the two group members (step 120) and the pre-defined pattern. One example for carrying out this step is by dividing the audio spectrum into a plurality of frequency ranges (bands), finding the best hearing capability for each one of these frequency ranges (from among the data available for the different group members), and determining the deviation (gap) that exists at each of the frequency ranges between the best hearing capability for that range and a pre-defined hearing pattern. Although this option would result in minimizing the modification that will be affected for each frequency range, still, it will result in less distortion for members whose hearing capabilities have not substantially deteriorated. Another processing option may be for example, averaging the gaps that exist between the hearing capabilities associated with the different group members, and pre-defined values which characterize a perfect hearing capability at each respective frequency range.

Once this determination process has been completed, a compensation algorithm is applied (step 130) in order to determine how should the audio signals be modified, so that they when they are played to the group members it will be done in a way that the audio signals would sound pleasant to the various members of the group.

It should be noted that determining the required compensation is typically a complex process, which preferably takes into account different frequencies and bands (in terms of the range(s) of frequencies affected) and does not simply create a “mirror” image of the common audiogram (i.e. being the audiogram that takes into account the at least two members of the group). Instead, it is preferably used to generate a smooth and enjoyable equalized curve, and optionally, allows to compensate differently tones that will be heard by the left ears from the compensation of tones as will be heard by the right ears. For example, if an individual has a 3 db drop at around 500, it might be more musically pleasing to enhance the frequencies around 500 by 2.5 db through a broadband curve extending from 450 Hz to 570 Hz, rather than just bumping the 500 drop by 3 db. Another option could be for example to reduce the level of few frequencies around the 500 Hz frequency instead of bumping the signal at the 500 Hz frequency.

According to the present example, the analysis results of the common audiogram are compared by executing a compensation algorithm with a pre-defined pattern (e.g. a straight line having a volume equal to zero throughout the whole frequencies' spectrum that was checked in the test). Then the compensating algorithm, which may include equalization process, determines the frequency ranges at which the audio signals will be modified by applying the appropriate compensation associated with the group's common audiogram. In other words, the compensating algorithm uses predetermined equalizing (EQ) bands and curves to determine the compensation that will be provided to modified audio signals for the group, a compensation which may be heard either differently by the members' left ears or right ears, or the same.

When the members of the group decide to listen to music, whether by using a new music player or by using a music player that had already been used by that group before, the data resulting from the compensation algorithm is provided to two customized equalizers (for the L/R ears) of the music player in such a way that the audio signals may be modified in each of the two channels accordingly. The data may be retrieved from a storage comprised in the music player, if that music player has already been used before by that group of listeners, but provided that the music player comprises a storage means. In the alternative, the data may be retrieved on an ad-hoc basis from the remote server or from the members' smartphones, depending on where the relevant data has been stored, irrespective of whether this is a new music player or one that had already been used.

The equalization data (i.e. frequency, volume and “Q” value) are forwarded to a DSP comprised within the music player. The value of parameter “Q” referred to above, relates to the angle and slope of the gain given for each frequency band. It may be very sharp and bell shaped, thus affecting only a rather narrow range of frequencies, or on the other hand, it may be quite flat and large, thus affecting a larger range of frequencies.

Although the compensation process described in this example relates to implementing a different compensation for left and right channels where the music will be played for the group members, it should be clear that the present invention also encompasses cases where the compensation is made only for one ear, or for cases where essentially the same compensation is applied for the group members, irrespective of the channel (left or right) via which the music will be played. In the latter case, the hearing test which was taken by each of the at least two members, could have been one test where both ears were tested simultaneously thereby receiving a combined result, or that the ears were tested separately but then the results obtained for each of the two ears, were combined in the process of determining a compensation that will be applied while modifying the audio signals.

The above exemplified method also offers the freedom for a certain individual who has already taken such a hearing test and has the data the relates to his/her hearing capabilities stored, to join any different group of individuals listening to modified audio signals. The data associated with that individual will then be retrieved when a group of people, including that individual, decide to listen to music (audio signals), followed by establishing a common audiogram for that specific group, one that includes also relevant data associated with this individual.

Following the determination of how to modify the audio signals, the modified audio signals are played to the members of the group of individuals (step 140).

As described hereinabove, in order to carry out the present invention, data associated with the hearing capabilities of at least two of the group members (and not necessarily all group members), is utilized. However, according to another embodiment, the compensation algorithm also takes into account the number of the group members for whom there is available data associated with their hearing capabilities, as opposed to the total members who currently belong to that group. This fact is taken into account as there is likely to be, according to this embodiment, a different conclusions (and consequently a different compensation) if the data is known for all group members, in which case the compensation may be more tailored to these members, as opposed to cases where data is known only for few members (e.g. two members of say a five members' group), and consequently the compensation algorithm takes into account not to modify/compensate the audio signals too aggressively, as otherwise, one or more of the members (for whom no data is available) might find the modified audio signals too grating (annoying) for them to listen.

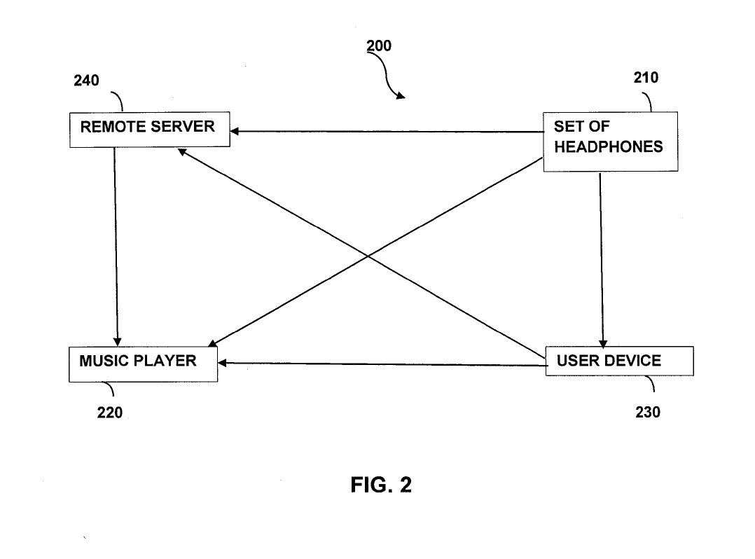

FIG. 2 is a block diagram presenting a system 200 for implementing one or more embodiments of the present invention.

System 200 of the example illustrated in this FIG., comprises the following components:
A set of headphones 210 (or a number of such sets) that may be used for carrying out the hearing tests as described above, to at least two listeners who will eventually belong to the same group that is about to listen together to music;
A music player 220 such as a music center, a car's radio center, etc.;
An optional user device 230 such as a smartphone, tablet, etc., which may be used in some of the embodiments described hereinafter; and
An optional remote server 240, which may be used in some of the embodiments described hereinafter.

The set of headphones 210 comprises a sound generator configured to generate tones that will be heard by an individual who is taking the hearing test and a processor configured to determine the results of the hearing test. In addition, the set of headphones comprises a transmitter (e.g. a cellular transmitter, or a Wi-Fi transmitter or a Bluetooth transmitter and the like) that is operative to forward the results of the hearing tests. The results that will be forwarded may be in a form of raw results obtained during the test, or in the alternative, they can be processed by the processor comprised in the set of headphones, so that the data that will be forwarded by the headphones' transmitter will already be in a processed form, (e.g. identifying one or more frequencies where there is a deviation of the hearing capability of the individual taking the test from a pre-defined pattern and optionally its magnitude).

According to the present example, the hearing test related data that should be forwarded from the set of headphones, possibly towards any one of the other three components of system 200, depending on the embodiment by which the implementation will be carried out.

According to one group of embodiments, the hearing test related data of the at least two group members may be forwarded instantaneously to music player 220 (e.g. by using a Wi-Fi/Bluetooth protocols, if the set of headphones used by the at least two individuals taking the test and the music player are both located at a geographical proximity, to allow using such a data transfer mechanism), or it may be stored at the set of headphones until the set of headphones and the music player are brought to a geographical proximity, which in turn would allow initiating such a data transfer. The data transfer to the music player will be carried out either automatically, or be initiated by one of the group members.

According to another group of embodiments, the hearing test related data of the at least two group members, may be forwarded and uploaded to a user device 230 (such as a smartphone) or to a number of user devices. In the latter case each user device is adapted to store the results of the individual who is associated (e.g. owns) with that user device. This may be carried out either immediately (e.g. by using a Wi-Fi/Bluetooth protocols if the smartphone and the set of headphones are both located at a geographical proximity), or it may be stored at the set of headphones, and when the set of headphones and the smartphone are brought to a geographical proximity with each other allowing such a data transfer, the data transfer to the smartphone will be carried out either automatically, or be initiated by an individual (e.g. by activating a proper application installed at his/her smartphone).

Once the data has been uploaded to the one or more smartphones, there are again two options of how to proceed with the process. By the first option, the data will be forwarded, e.g. over the Internet, to a remote server for storage, while by the second option, the data will be forwarded to the music player. As before, if the data arriving at the smartphone (the user device) is in a form of raw test results (or even partially processed), they may either be processed (or partially processed) by the processor of the smartphone itself as described above, and then be forwarded as processed, or the data may be further forwarded as received.

In the case that data is transferred directly from the smartphone to the music player, it can be done either on a one-time basis, where data is transferred to the music player and stored thereat. This option ensures that whenever that individual joins a group of members who will be using that music player, he/she will be identified and his/her hearing capabilities related data may be easily taken into account when determining how the audio signals should be modified to suit the group members' hearing capabilities. In the alternative, the data may be stored at each of the at least two individuals' smartphones, and when a group of people would like to listen together to music played by any music player is formed on an ad hoc basis, each of the at least two smartphones will provide the music player with the data required in order to establish the common audiogram which in turn would allow to modify the audio signals in accordance with the at least two individuals' hearing capabilities.

According to still another group of embodiments, the hearing test related data may be forwarded and uploaded at the remote server 240. It can be done either directly from the set of headphones 210 (e.g. by using a cellular modem) or from smartphone 230, following the uploading of the data at that smartphone. In this group of embodiments, once at least two individuals have indicated that they both wish to listen to a music in a group mode while using a certain music player, that indication would be forwarded to the remote server (e.g. by way of a message specifying the identity of all individuals who are currently interested in establishing a group for listening together to music, and preferably the identity (e.g. the IP address) of the music player to which data associated with the identified individuals that is about to be retrieved from the storage means of the remote server, should be sent.

There is a number of options that may be implemented in system 200 to enable individuals to indicate their wish to listen together to music in a group mode, where that music has been modified according to the hearing capabilities of at least two group members. One such option is a user interface installed at the music player (e.g. a button or a set of buttons), that once used, a message is generated and sent either to the remote server or to all identifiable smartphones at the geographical proximity of the music player, depending on where the data that is required by the music player processor for carrying out the modifications, is stored. Another option is that a message is initiated by a group member by typing a pre-defined command, using the smartphone keyboard. In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention in any way. For example, although the processing of the data characterizing the various group members into one common set of data, has been described in quite a few examples provided throughout the specification as taking place at the remote device, the present invention should be understood to encompass also a variation wherein this processing is carried out by forwarding sets of data, each describing a group members, from the remote device to the music player, and processing the sets of the data into ne common set of data for modifying the audio signals for the group is carried out by the music player. The described embodiments comprise different features, not all of which are required in all embodiments of the invention. Some embodiments of the present invention utilize only some of the features or possible combinations of the features.

Variations of embodiments described herein and embodiments of the present invention comprising different combinations of features noted in the described embodiments will occur to persons of the art. The scope of the invention is limited only by the following claims.

1. A method for modifying audio signals for a group of listeners in accordance with hearing capabilities of at least two members belonging to a group of listeners, the method comprises the steps of:
providing information that relates to hearing capabilities of the at least two members belonging to a group of listeners;
based on the information provided, determining respective values for one or more parameters to be applied for modifying audio signals that are about to be played, in a way that would suit all members belonging to said group of listeners;
modifying said audio signals by applying the respective values of the one or more determined parameters to modify said audio signals; and
enabling playing the modified audio signals to all members of said group of listeners.

2. The method of claim 1, wherein the information that relates to the hearing capabilities of the at least two members belonging to a group of listeners, comprises deviations in the hearing capabilities of the at least two members from a pre-defined hearing pattern.

3. The method of claim 1, wherein the step of determining the one or more parameters for modifying audio signals comprises averaging gaps that exist in a plurality of audio tones as they were actually heard by each of the at least two members of the group, and the plurality of audio tones as they should have been heard by these at least two members, had they both had a perfect hearing capability.

4. The method of claim 1, further comprising selecting on an ad hoc basis who will be the members of the group of listeners, prior to playing the modified audio signals to the members that would eventually belong to that group.

5. The method of claim 1, wherein at least one of the members of the group of listeners is a member of at least one other group of listeners.

6. The method of claim 1, wherein the step of providing information comprises providing information that relates to hearing capabilities of a first plurality of members belonging to the group of listeners, and determining the one or more parameters for modifying audio signals based on information that relates to hearing capabilities of a second plurality of members selected from among said first plurality of members.

7. The method of claim 1, wherein at least one of the one or more parameters has at least two different values, each associated with a respective range of frequencies.

8. The method of claim 1, wherein the step of providing information comprises providing information that relates to hearing capabilities of each ear of the at least two members who belong to the group of listeners.

9. The method of claim 1, further comprising a step of determining at least one range of frequencies at which input audio signals will be modified, and the modification of the input audio signals that will be carried out for audio signals belonging to said range of frequencies.

10. A music player configured to modify audio signals when played to a group of listeners in accordance with hearing capabilities of at least two members belonging to said group of listeners, said music player comprising:
at least one processor configured to:
(i) receive information that relates to hearing capabilities of the at least two members belonging to the group of listeners; and
(ii) modify audio signals to be played to said group of listeners by taking into account the hearing capabilities of the at least two members belonging to the group of listeners, in a way that would suit all members belonging to said group of listeners;
a sound generating module operative to play the modified audio signals.

11. The music player of claim 10, wherein the at least one processor is further configured to determine, based on the information received, respective values for one or more parameters to be applied for modifying audio signals that are about to be played in a way that would suit all members belonging to said group of listeners.

12. The music player of claim 10, further comprising a receiver configured to receive information from another electronic device, wherein the information relates to the hearing capabilities of the at least two members of the group of listeners.

13. The music player of claim 12, wherein the receiver is operative to receive the information that is wirelessly transmitted thereto.

14. The music player of claim 12, wherein the other electronic device is a server located remotely from the music player and configured to store the information that relates to the hearing capabilities of the at least two members of the group of listeners.

15. The music player of claim 10, wherein the music player further comprises a user interface configured to enable receiving a command initiated by or on behalf of at least one member of the group of listeners, wherein the command comprises identification of at least one listener who belongs to said group of listeners.

16. The music player of claim 12, wherein the music player further comprises a transmitter operative to transmit a command received by the music player from or on behalf of at least one member of the group of listeners towards the other electronic device, instructing said other electronic device to forward the information that relates to hearing capabilities of the at least two members of the group of listeners, towards the music player.

17. The music player of claim 10, further comprising a storage configured to store information that relates to hearing capabilities of the at least two members of the group of individuals.

18. The music player of claim 17, wherein the processor is further configured to retrieve information that relates to hearing capabilities of the at least two members of the group of listeners from said storage, and apply it for modifying the audio signals that are about to be played to said group of listeners.

19. A computer program product encoding a computer program stored on a non-transitory computer-readable medium for executing a set of instructions by one or more computer processors for carrying out the method of claim 1.
